(19) United States
(12) Patent Application Publication
(10) Pub. No.: US 2026/0276617 A1
(43) Pub. Date: Sep. 17, 2026

(54) Modular Water Quality System with Wifi and Variable Power Sources

(71) Applicant: Spencer Perry, Gainesville, GA (US)

(72) Inventor: Spencer Perry, Gainesville, GA (US)

(21) Appl. No.: 19/667,510

(22) Filed: May 4, 2026

Related U.S. Application Data
(60) Provisional application No. 63/799,669, filed on May 4, 2025.

(51) Int. Cl. G01N 33/18; G01N 37/00

(52) U.S. Cl. CPC G01N 33/18; G01N 37/00

(57) ABSTRACT

This water quality monitoring system uses a microcontroller connected to interchangeable sensors that simultaneously measure pH, turbidity, dissolved solids, temperature, water level, flow rate, oxidation-reduction potential, and soil moisture. It automatically detects and bypasses faulty sensors without disrupting overall operation. Raw data is filtered and calibrated, then converted into practical indicators like a Water Quality Index, corrosion risk, sediment runoff risk, and algae growth potential. A GPS module tags each reading with location and timestamp. Data is accessible three ways: an LCD screen, SD card (CSV format), and a wireless web server with remote update support. Color-coded alerts and audible warnings make results easy to interpret for non-experts. The system runs on solar, battery, or AC power for remote deployment. One-button calibration and a single-unit design cut costs to roughly one-tenth of comparable commercial systems.

### BACKGROUND OF THE INVENTION

Access to safe drinking water represents a critical global challenge, with over two billion people lacking access to safely managed drinking water, primarily in underserved communities in Africa and Asia. In these regions, environmental hazards and natural disasters that affect water quality are frequently detected too late for an effective response due to limited or nonexistent water quality monitoring infrastructure.

Existing water monitoring solutions present significant barriers to widespread adoption. Current multi-parameter monitoring systems are prohibitively expensive, often costing approximately $6,000 or more, making them inaccessible to individual users, community organizations, and resource-limited municipalities. Additionally, existing systems are difficult to deploy at scale, and field repair or replacement is often impractical in remote or disaster-affected environments. Natural disasters frequently disable existing monitoring systems entirely, leaving affected communities without critical water safety data precisely when it is needed most. Actionable water quality data is frequently inaccessible to non-expert users, requiring specialized hardware, software, or internet connectivity to interpret raw sensor output. Furthermore, undetected sensor failures in unattended monitoring systems can silently compromise long-term water quality data integrity without alerting operators.

These limitations were demonstrated following Hurricane Helene's destruction across the southeastern United States, which caused unprecedented flooding in Appalachian mountain communities, destroying water treatment infrastructure and introducing pollutants into water supplies across a wide geographic area. Evaluating water quality damage required manual sample collection across thousands of test sites, demanding extensive human resources and causing dangerous delays in water safety assessment. Similar resource limitations affect local water monitoring organizations operating near bodies of water such as Lake Lanier, Georgia, where funding constraints restrict access to expensive commercial monitoring systems despite ongoing water quality concerns.

There exists a need in the art for a low-cost, modular, self-diagnosing water quality monitoring system capable of autonomous multi-parameter sensing, real-time fault detection, and human-centered data interpretation accessible to non-expert users in resource-limited and disaster-affected environments.

### BRIEF DESCRIPTION OF THE DRAWINGS

FIG. 1 shows a flowchart illustrating the operational sequence of the water quality monitoring system from initial power-up (100) through optional network configuration (101), sensor calibration (102), and device placement (103), branching into two operational modes: continuous remote deployment (104) with data accessible via web server (106) or SD card (107), and immediate on-site monitoring (105) with readings displayed on the integrated digital screen (108) and alerts for out of range conditions (109).

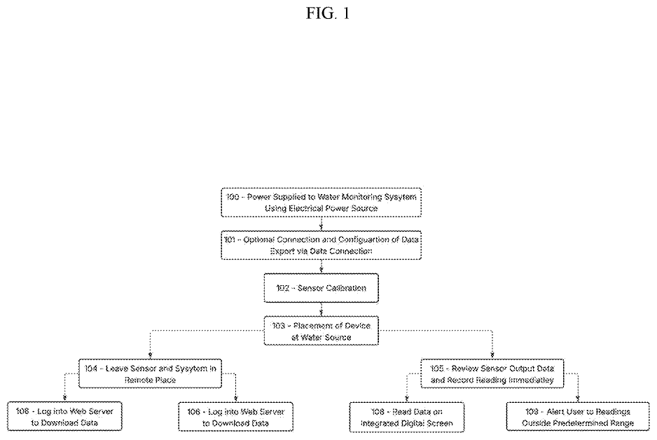

FIG. 2 shows a system architecture diagram illustrating the central ESP32 microcontroller (209) communicatively coupled to eight modular, non-proprietary sensors (201-208) measuring total dissolved solids, temperature, soil moisture, oxidation-reduction potential, water level, pH, flow rate, and turbidity, a GPS module or similar position data sensor (210) for geographic and temporal tagging, and a power supply subsystem (211) comprising solar panel, battery, and adapter options, with the microcontroller driving four output modalities: a web server (212), SD card (213), LCD screen (214), or predetermined alert functions based on data ranges (215).

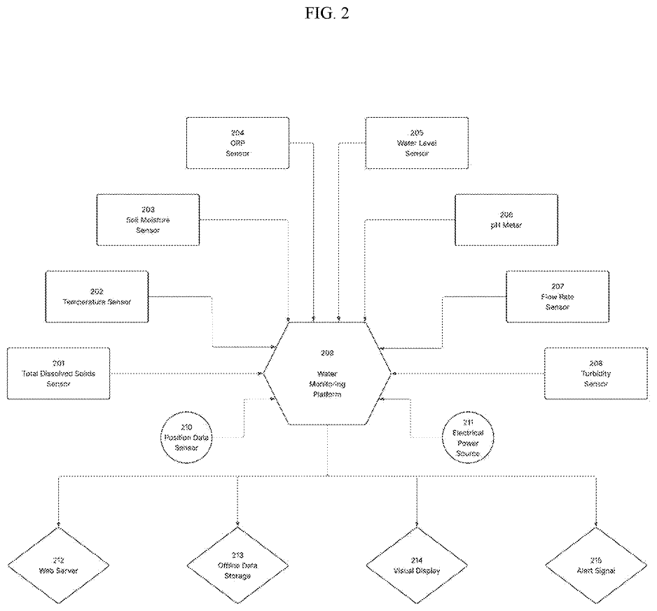

### DEFINITIONS

Unless otherwise stated, the following definitions apply throughout the specification and the appended claims:

“Non-proprietary sensor” means a sensor that is commercially available from multiple sources and is not exclusively tied to a single manufacturer's system or interface standard, such that equivalent replacement units may be sourced and installed without licensing restrictions or specialized proprietary tooling.

“Modular” means configured for individual physical removal and replacement independently of other system components and without requiring disassembly of the system as a whole.

“Derived metric” means a value calculated by the microcontroller from one or more raw or filtered sensor measurements using a defined mathematical relationship, which value is not itself directly measured by any single sensor.

“Advanced environmental insight” means a classification or assessment of an environmental condition calculated by the microcontroller from one or more sensor values or derived metrics and expressed as a severity classification for presentation to a user.

“Severity classification” means one of a defined set of user-facing output categories, namely Warning, Caution, and Safe, assigned to a sensor reading or advanced environmental insight based on comparison of a computed value against predefined thresholds and intended to convey actionable information to a non-expert user without requiring interpretation of underlying numerical data.

“Fault-tolerant” means capable of continuing normal system operation, including sensor data acquisition, derived metric calculation, advanced insight calculation, and data output, in the presence of one or more malfunctioning or excluded sensors.

“Over-the-air firmware update” means a process by which updated firmware code is transmitted to the microcontroller via a wireless network connection and installed without requiring physical access to the deployed unit or connection of any external programming device.

“Frozen value” means a condition wherein a sensor reports an identical or substantially identical output value across a plurality of consecutive sampling intervals during which other sensors in the array report normally varying values, indicating a sensor malfunction rather than a true environmental condition.

“Resource-limited environment” means a geographic or operational context characterized by restricted access to electrical grid power, internet connectivity, specialized technical personnel, or commercial monitoring equipment, including but not limited to rural and remote communities, post-disaster areas, and developing-region municipalities.

### DETAILED DESCRIPTION

The present invention comprises a low-cost, modular, self-diagnosing, multi-parameter water quality monitoring system configured for autonomous operation in resource-limited, remote, and disaster-affected environments. The system integrates a plurality of non-proprietary, interchangeable sensors with a microcontroller, a signal processing and fault-detection architecture, a human-centered data interpretation layer, multiple power supply options, and multiple data output modalities. The system is capable of simultaneous measurement of up to eight water quality parameters, autonomous fault detection and sensor exclusion, local computation of derived metrics and advanced environmental insights, and data transmission or storage without requiring external network connectivity for core monitoring functions. The system is designed to be deployable at approximately one-tenth the cost of comparable commercial multi-parameter monitoring systems, enabling adoption by individual users, community organizations, emergency response personnel, and resource-limited municipalities in underserved and disaster-affected regions.

The water quality monitoring system is initialized by connecting a power supply (100) to the system. The power supply subsystem (211) comprises one or more of a 12-volt solar panel, an alkaline or lithium polymer battery, and a standard wall adapter, enabling deployment across resource-limited environments without access to grid power. The availability of multiple power supply options is a particular advantage in disaster-response scenarios where grid infrastructure has been disrupted and in remote field settings where continuous autonomous monitoring would otherwise be impractical.

In one embodiment, the central computing element of the system is an ESP32 microcontroller (209), which manages all sensor data acquisition, signal processing, diagnostic evaluation, derived metric calculation, advanced environmental insight generation, and data output operations. In this embodiment, eight modular, non-proprietary sensors are communicatively coupled to the ESP32 microcontroller (209): a Total Dissolved Solids sensor (201) measuring dissolved ionic content in parts per million; a Temperature sensor (202) measuring water temperature in degrees Celsius; a Soil Moisture sensor (203) measuring ground saturation percentage; an Oxidation-Reduction Potential sensor (204) measuring electrochemical oxidative or reducing conditions in millivolts; a Water Level sensor (205) measuring water level percentage; a pH sensor (206) measuring chemical balance on the standard pH scale; a Flow Rate sensor (207) measuring volumetric flow in liters per minute; and a Turbidity sensor (208) measuring suspended particulate concentration. Because each sensor is both modular and non-proprietary, any individual sensor may be sourced from multiple commercial suppliers and replaced in the field without disassembly of the system as a whole, without licensing restrictions, and without specialized proprietary tooling, a combination of properties that is particularly advantageous in resource-limited environments where supply chains and technical personnel may be unavailable.

In another embodiment, a GPS module (210) is coupled to the ESP32 microcontroller (209) and it records the geographic coordinates as well as a UTC timestamp of each data collection session, enabling spatial and temporal tagging of all measurements. This feature supports distributed multi-unit deployments in which data from multiple systems may be aggregated across a geographic area for watershed-scale or municipal-scale environmental monitoring. System on chip integrated circuits are capable of supporting a global positioning system in tandem with multiple probes.

Prior to deployment, the user may optionally connect the system to a WiFi network (101) to enable remote data access via the web server (212). WiFi connectivity is not required for system operation; in embodiments where network access is unavailable or not desired, the system operates autonomously in a local-only mode without loss of any sensing, processing, or local output functionality. The user then performs one-button sensor calibration (102), wherein offset values are applied to the connected sensors to account for environmental baseline conditions and sensor-specific drift. Following calibration, the sensor array (103) is placed in the target water source.

The system supports two primary operational modes. In a continuous autonomous deployment mode (104), the system collects and records sensor data at regular one-minute intervals without user intervention, transmitting data to the web server (212) over WiFi where connectivity is available, and concurrently logging all sensor readings, derived metrics, advanced environmental insights, GPS coordinates, and timestamps to an SD card (213) in CSV format. Data logged to the SD card (213) is retrievable (107) for offline analysis and may be exported to external water management or decision-support systems. In an immediate on-site monitoring mode (105), real-time sensor readings and advanced environmental insights are presented directly on the LCD screen (214), enabling field assessment without requiring network connectivity or subsequent data retrieval.

In either operational mode, the alert (215) activates automatically when any measured parameter receives a severity classification of Warning (109), providing immediate notification to non-expert users without requiring interpretation of underlying numerical sensor data. This alert mechanism operates independently of network connectivity, ensuring that warning capability is preserved in fully offline deployments in resource-limited environments.

The ESP32 microcontroller (209) calculates a plurality of derived metrics from raw and filtered sensor measurements, including Conductivity, Water Quality Index, Water Stability Index, Organic Load, Thermal Comfort, Microbial Growth potential, Dissolved Oxygen status, Dissolved Oxygen concentration, Chlorine Residual estimation, and Ammonia State assessment. From these derived metrics and validated sensor values, the microcontroller (209) further calculates a plurality of advanced environmental insights, including Corrosion Risk, Scaling Risk, Disinfection status, Aerobic State, Algal Bloom risk, Sediment Runoff risk, Salinity Event detection, Flooding risk, Drought conditions, Leak detection, Shortage detection, Temperature Stress, Irrigation Aid suitability, and Clarity assessment. Each advanced environmental insight is assigned a severity classification of Warning, Caution, or Safe based on comparison of the computed value against predefined thresholds, and is presented to the user through one or more output modalities in a form that conveys actionable information without requiring the user to interpret the underlying numerical data. This human-centered interpretation layer is a particular advantage of the invention, as it makes the system accessible to non-expert users including community organizations, municipal workers, and disaster-response personnel operating in resource-limited environments.

The system is fault-tolerant with respect to individual sensor failures. The ESP32 microcontroller (209) continuously evaluates each sensor in the array for frozen values, out-of-range readings, disconnection, and calibration drift. A sensor reporting a frozen value, that is, an identical or substantially identical output across a plurality of consecutive sampling intervals during which other sensors report normally varying values, is identified as malfunctioning and automatically excluded from data output and from derived metric and advanced environmental insight calculations. This exclusion occurs without halting operation of the remainder of the system, such that the system continues to acquire data, calculate metrics and insights from valid sensors, and transmit or log output through all available modalities notwithstanding the excluded sensor. Fault status for each sensor is reported to the user through the web server (212) and LCD screen (214), enabling informed assessment of data integrity without specialized diagnostic equipment.

Where WiFi connectivity is available, the web server (212) further supports over-the-air firmware updates, by which updated firmware code is transmitted to the ESP32 microcontroller (209) via the wireless network connection and installed without requiring physical access to the deployed unit or connection of any external programming device. This capability is a particular advantage in remote or resource-limited deployments where physical retrieval of the unit for firmware maintenance would be impractical.

1. A water quality monitoring system comprising:
A microcontroller configured to receive sensor data, perform signal processing, execute diagnostic evaluations, calculate derived metrics and advanced environmental insights, and manage data output;
a plurality of modular, non-proprietary sensors communicatively coupled to the microcontroller, the sensors measuring two or more of total dissolved solids, temperature, soil moisture, oxidation-reduction potential, water level, pH, flow rate, and turbidity;
a GPS module communicatively coupled to the microcontroller and configured to record geographic coordinates and timestamps associated with each sensor measurement session;
a power supply subsystem comprising two or more of a solar panel, a battery, and a wall adapter; and
one or more output modalities communicatively coupled to the microcontroller and comprising two or more of a web server, an SD card data logger, an LCD display, and an audible alert device.

2. The system of claim 1, wherein the plurality of modular, non-proprietary sensors comprises a total dissolved solids sensor, a temperature sensor, a soil moisture sensor, an oxidation-reduction potential sensor, a water level sensor, a pH sensor, a flow rate sensor, and a turbidity sensor.

3. The system of claim 1, wherein the microcontroller is configured to perform signal processing by applying hardware RC filtering to raw sensor signals to attenuate high-frequency electrical noise, and applying software median filtering to RC-filtered signals to eliminate transient spike outliers prior to diagnostic evaluation.

4. The system of claim 1, wherein the microcontroller is configured to execute diagnostic evaluations by detecting, for each sensor in the plurality of modular, non-proprietary sensors, one or more of frozen values, out-of-range readings, disconnection, and calibration drift, and to automatically exclude any sensor determined to be malfunctioning from data output and from derived metric and advanced environmental insight calculations without halting operation of the remaining system.

5. The system of claim 1, wherein the derived metrics calculated by the microcontroller comprise two or more of Conductivity, Water Quality Index, Water Stability Index, Organic Load, Thermal Comfort, Microbial Growth potential, Dissolved Oxygen status, Dissolved Oxygen concentration, Chlorine Residual estimation, and Ammonia State assessment.

6. The system of claim 1, wherein the advanced environmental insights calculated by the microcontroller comprise two or more of Corrosion Risk, Scaling Risk, Disinfection status, Aerobic State, Algal Bloom risk, Sediment Runoff risk, Salinity Event detection, Flooding risk, Drought conditions, Leak detection, Shortage detection, Temperature Stress, Irrigation Aid suitability, and Clarity assessment, each expressed as a severity classification assigned based on comparison of a computed value against predefined thresholds.

7. The system of claim 1, wherein the web server is configured to receive and install over-the-air firmware updates transmitted to the microcontroller via a wireless network connection without requiring physical access to a deployed unit.

8. The system of claim 1, further comprising a plurality of water quality monitoring systems networked together, wherein data collected from the plurality of systems is aggregated to generate simulations of water quality conditions across a geographic area.
